(19) United States
(12) Patent Application Publication
(10) Pub. No.: US 2016/0296764 A1
(43) Pub. Date: Oct. 13, 2016

(54) NON-INVASIVE AND NON-ABLATIVE SOFT TISSUE LASER THERAPY

(71) Applicant: Gary John Bellinger, Dallas, TX (US)

(72) Inventor: Gary John Bellinger, Dallas, TX (US)

(21) Appl. No.: 14/789,958

(22) Filed: Jul. 1, 2015

Related U.S. Application Data
(60) Provisional application No. 62/019,702, filed on Jul. 1, 2014.
(60) Provisional application No. 62/019,708, filed on Jul. 1, 2014.

(51) Int. Cl. A61N 5/06

(52) U.S. Cl. CPC A61N 5/0616; A61N 2005/0644; A61N 2005/0659; A61N 2005/067

(57) ABSTRACT

A laser irradiation system, method, and apparatus that can generate optical energy at a specific or a range of wavelengths, power levels, and beam profiles, among others, to treat acute or chronic inflammation, wounds, and autoimmune deficiency conditions without ablating the target tissue or surrounding tissue. In one aspect, the light beam of the laser irradiation system, method, and apparatus can stimulate photoreceptors within a cell, thereby initiating a cascade of secondary cellular metabolic effects and normalizing cellular activity towards homeostasis, among other advantages.

### CROSS-REFERENCE TO RELATED APPLICATIONS

This application claims the benefit of U.S. Provisional Application No. 62/019702 filed on Jul. 1, 2014, which is incorporated herein by reference in its entirety, and U.S. Provisional Application No. 62/019708 filed on Jul. 1, 2014, which is incorporated herein by reference in its entirety.

### BACKGROUND

This section is intended to introduce the reader to aspects of art that may be related to various aspects of the disclosure described herein, which are described and/or claimed below. This discussion is believed to be helpful in providing the reader with background information to facilitate a better understanding of the various aspects of the disclosure described herein. Accordingly, it should be understood that these statements are to be read in this light, and not as admissions of prior art.

Several non-surgical methods have been utilized in the therapeutic treatment of acute or chronic inflammation in living tissue. Some of the techniques previously utilized included the application of ultrasonic energy, electrical stimulation, high frequency stimulation by diathermy, X-rays and microwave irradiation. Techniques such as electrical stimulation, diathermy, X-ray and microwave radiation have shown some therapeutic benefit for soft tissues, however, their use has been somewhat limited because of tissue damage caused by excessive thermal effects. Consequently, the energy levels associated with therapeutic treatments involving diathermy, X-ray, microwave, and electrical stimulation have been limited to such low levels that have little or no benefit for treating acute or chronic inflammation, wounds, or joint pain. Additionally, the dosage of exposure to microwaves and X-ray radiation must be carefully controlled to avoid radiation related health problems. Ultrasonic energy is non-preferentially absorbed and can negatively affect all of the surrounding or otherwise healthy tissue.

In addition, optical energy generated by lasers has been applied for various medical and surgical purposes because of the monochromatic and coherent nature of laser light which can be selectively absorbed by living tissue depending upon certain characteristics of light of variable wavelengths and also the properties exhibited by viable cells in the irradiated tissue such as reflectivity, absorption coefficient, scattering coefficient, thermal conductivity, and thermal diffusion constant. The reflectivity, absorption coefficient, and scattering coefficient are dependent upon the wavelength of the optical radiation. Further, the absorption coefficient is known to depend upon such factors as inter band transition, free electron absorption, grid absorption (phonon absorption), and impurity absorption, which are dependent upon the wavelength of the optical radiation. Conventional lasers using low power or low level lasers using wavelengths that do not allow for any type of significant penetration into the body because of the absorption of the energy into melanin, hemoglobin, and oxy-hemoblogin. The wavelengths used by these low power and low level lasers prevent the body from absorbing the laser energy for chronic or acute inflammation or wound healing benefits.

Water is a major component of living tissue, which has an absorption band according to the vibration of water molecules in the infrared range. In the visible range, absorption exists due to the presence of hemoglobin. Additionally, the scattering coefficient in living tissue is a dominant factor. Therefore, for a specific tissue type, the laser light may propagate through the tissue substantially unattenuated, or may be almost entirely absorbed. The extent to which the tissue is heated and ultimately destroyed depends on the extent to which it absorbs the optical energy. It is generally preferred that the laser light be essentially transmissive in tissues that are desired not to be affected, and absorbed by the tissues which are to be affected. For example, when applying laser radiation in a tissue field that is wet with blood or water, it is desired that the optical energy not be absorbed by the water or blood, thereby permitting the laser energy to be directed specifically to the tissue to be treated.

Hence, what is needed is a laser irradiation system, method, and apparatus that can generate optical energy at specified or range of wavelengths, power levels, and beam profiles, among others, to treat acute or chronic inflammation, wounds, and autoimmune deficiency conditions, among others, without ablating the target tissue or surrounding tissue.

### BRIEF SUMMARY

In one aspect of the disclosure described herein, a laser irradiation system, method, and apparatus is provided that can generate optical energy at a specific or a range of wavelengths, power levels, and beam profiles, among others, to treat acute or chronic inflammation, wounds, and autoimmune deficiency conditions, among others, without ablating the target tissue or surrounding tissue. More specifically, the laser irradiation method and system can percutaneously stimulate biological or cell tissue, in a non-discriminatory fashion and intracellularly, until energy homeostasis is achieved. In particular, the laser beam and optical energy of the disclosure described herein does not significantly absorb into melanin, hemoglobin, or oxy-hemoglobin. Here, the power of the laser delivered to a patient or treatment site is substantially high and dense, thereby allowing greater depths of laser light penetration and achieve higher and accelerated wound healing or inflammation healing results as compared to conventional low power laser light systems and methods. In addition, the beam profile of the disclosure described herein can be de-focused and distanced from the skin of a patient thereby preventing tissue damage and enable healing. The system, method, and apparatus of the optical irradiation cell therapy of the disclosure described herein can effect cellular activity by stimulating cell growth, increasing cell metabolism, improve cell regeneration, invoke an anti-inflammatory response, promote edema reduction, reduce fibrous tissue formation, stimulate nerve function, and stimulate the production of endorphins, among other advantages. This can further result in improve blood flow and vascularization in damaged tissue, improve and restore function of nerve cells in damaged tissue, produce natural opiates and other compounds that reduce pain and simulate healing, and direct stimulation of cellular grown and healing of soft tissues such as collagen.

In another aspect of the disclosure described herein, the laser system, method, and apparatus can include laser light wavelengths, power, and beam profiles which are optimal for penetrating the skin and getting deep into injuries as the laser light passes through cell tissue. Hence, the deep penetration of the laser light of the disclosure described herein minimizes the scatter of the laser energy by decreasing the loss of energy, and avoids excessive heat and discomfort for a patient. In addition, the laser system, method, and apparatus can operate with energy levels from approximately, 5 to 100 Watts, preferably 20 to 70 Watts. These high energy levels can further result in higher penetration and stronger biostimulation of cell tissue. The laser can safely deliver approximately 50 to 200 times the amount of continuous laser energy to damaged tissues below the skin's surface than other lower wavelength, non-thermal laser therapy devices. Further, the laser system, method, and apparatus can treat large treatment sites or large area of damaged tissue more efficiently and more uniformly because the optical components can create a laser beam profile of up to approximately 60 centimeters in diameter, preferably 30 cm, which is far larger than conventional laser systems having a 0.2 centimeter diameter. Further, the beam profile diameter can be adjusted to produce smaller diameter beams to treat smaller areas if needed which provides flexibility and a broader range of treatment options. Here, the wider beam of the present disclosure described herein can increase the amount of energy that can be safely used, and increases the tissue surface area covered at each treatment.

In one aspect of the disclosure described herein, a treatment protocol or methodology for the laser system can be determined by the type of injury and rate of healing. For example, in one embodiment, a single chronic injury may typically require an approximately 10-15 minute session per patient, whereas treatment protocols for more extensive injuries, or for multiple injuries, may require up to 30 minutes per patient or per session. Acute injuries may require treatments varying from three sessions per week to as many as two treatments per day and may be completed within a few days, or within a few weeks. Treatments may also be intermittent, with two weeks of treatment followed by two weeks of no treatments, and then repeated until the chronic pain or condition is relieved, cured, or disappears. Certain chronic conditions may require periodic maintenance treatments to prevent a reversal in the condition.

The method of the laser therapy of the disclosure described herein can further include utilizing a hand piece or hand-held portable unit having a laser source device and laser beam profile that is used for the irradiation of acute or chronically inflamed cells in a wound or tissue. Through the stimulation of photoreceptors within a cell, a cascade of intracellular metabolic reactions are initiated that move the cell towards homeostasis and in doing so resolves the acute or chronic inflammation in an expedited manner. The mechanisms include stimulating the expression and release of certain growth factors and cytokines from the cells that have infested the inflamed area, such as fibroblasts, macrophages, lymphocytes and endothelial progenitor cells. These biological mediators can bring about a proliferation of specific cell types within the wound, the inflamed tissue, or the wound or inflamed tissue's margins and coordinate the various stages of anti-inflammatory processes and wound healing, which then results in accelerated resurfacing of wounds or re-epithelialization and filling of the wound defect by granulation tissue and collagen with minimal or no scarring. The optical energy of the present disclosure described herein also increases the vascularity of the regenerating tissue that in turn results in more blood being brought to the inflamed, injured, or wound site and thus an increasing the rate of healing. Hence, there can be a resolution of metabolic deficits within cells far in excess of the effects of direct photo stimulation. Additionally, there is reduced scarring and control of superlative diseases of the skin. The laser therapy of the disclosure described herein offers an effective method, system, and apparatus for irradiating large numbers of cells with safe levels of optical energy to initiate the intracellular cascade toward homeostasis and the production of secondary effects contributing to homeostasis of adjacent or distant cells. This characteristic exponential benefit of photo stimulation is especially valuable in the treatment of acute or chronic inflammation and stimulation of the blood and immune cells of the body.

In one aspect of the disclosure described herein, a method of laser irradiation for alleviating the physical symptoms associated with acute or chronic inflammatory conditions is provided. The method can include focusing or aiming a light beam having a configured wavelength and power level on an inflamed area that is to be treated, wherein the light beam photo activates intracellular photoreceptors, thereby initiating a cascade of secondary cellular metabolic effects and normalizing cellular activity towards homeostasis. The method can further include treating surrounding reactive cells or viable cells near the inflamed area at identical time intervals so as to generate a preponderance of neutral or homeostatic cell responses en masse. In addition, the light beam can generate an increase in oxygenation in or around the inflamed area margins through angionesis or revascularization leading to wound healing or homeostatic cell response en masse. Here, the method can further include stimulating the production of intercellular messenger proteins and enzymes including superoxide dismutase and catalase enzymes. Here, the wavelength can include one or more wavelengths ranging from 1064 nm up to and including 1325 nm. The power level can further include one or more power levels ranging from 500 mW/cm² up to and including 5 W/cm². In addition, the light beam can further include a beam profile covering a surface area ranging from 0.1 cm² up to and including 60 cm². In addition, the light beam can also have a duration period from 30 seconds up to and including 3600 seconds. In addition, the light beam can further operate at a continuous wave mode. In addition, the light beam does not ablate cells within the inflamed treatment area or surrounding tissue area.

In another aspect of the disclosure described herein, a method of laser irradiation for alleviating the physical symptoms associated with acute or chronic inflammatory conditions isi provided. Here, the method can include positioning an optical source having infrared light adjacent to a treatment site, directing the infrared light at the treatment site, configuring a wavelength for the optical source, depending on the depth and type of inflammation at the treatment site, and determining a power level for the optical source, wherein the infrared light photo activates intracellular photoreceptors at the treatment site. In addition, the method can also include configuring a wavelength in the infrared spectrum between from 1060 nm to 1325nm. Also, the optical source may operate in power ranges from 750 mW/cm² to 1200 mW/cm², 350 mW/cm² to 1200 mW/cm², or 500 mW/cm² to 5 Watts/cm². The optical source can also be a hand-held unit.

In another aspect of the disclosure described herein, a method of laser irradiation for alleviating the physical symptoms associated with acute or chronic inflammatory conditions is provided. Here, the method can include directing a laser unit having infrared light at damaged or inflamed cells, configuring the laser unit to a wavelength of 1100 nm to 1275 nm and a time duration, depending on the depth and type of cells to be treated, wherein the depth can range from 0.1 cm to 15 cm, configuring the laser unit to a power level of 750 mW/cm² to 1200 mW mW/cm², and configuring the infrared light to a beam profile surface area of 1 cm² to 60 cm². In addition, the method can further include operating the laser unit in a continuous wave mode and having a homogenous beam profile. Also, the method can include treating the damaged or inflamed cells in a single treatment session for a duration of time ranging from 30 seconds to about 3600 seconds, and wherein the laser unit operates below the photo ablation threshold of the cells being treated.

The above summary is not intended to describe each and every disclosed embodiment or every implementation of the disclosure. The Description that follows more particularly exemplifies the various illustrative embodiments.

### BRIEF DESCRIPTION OF THE DRAWINGS

The following description should be read with reference to the drawings, in which like elements in different drawings are numbered in like fashion. The drawings, which are not necessarily to scale, depict selected embodiments and are not intended to limit the scope of the disclosure. The disclosure may be more completely understood in consideration of the following detailed description of various embodiments in connection with the accompanying drawings, in which:

FIG. 1 illustrates one non-limiting embodiment of a block diagram or flowchart describing the cellular regeneration process of treated cells.

FIG. 2 illustrates one non-limiting embodiment of a block diagram or flowchart describing the treatment process of a wound using the method of the disclosure described herein.

FIG. 3 illustrates one non-limiting embodiment front view diagram of a patient depicting a treatment method using one laser light source having one beam profile for treating a large treatment or surface area.

FIG. 4 illustrates another non-limiting embodiment front view diagram of a patient depicting a treatment method using plurality of laser light sources being used to treat a plurality of treatment sites.

FIG. 5 illustrates another non-limiting embodiment front view diagram of a patient depicting a plurality of overlapping laser beams focused on a treatment site.

FIG. 6 illustrates another non-limiting embodiment front view diagram depicting another treatment method in which a plurality of overlapping lasers beams focused on a plurality of overlapping treatment sites.

FIG. 7 illustrates one non-limiting embodiment perspective anterior view diagram of a shoulder area depicting a plurality of treatment points to be treated by the laser beam.

FIG. 8 illustrates another non-limiting embodiment perspective posterior view diagram of a shoulder area depicting a plurality of treatment points to be treated by the laser beam.

FIG. 9 illustrates a non-limiting embodiment cross-sectional view diagram of the subcutaneous tissue layers and depth of the laser beam extending beyond multiple soft and hard tissue layers.

FIG. 10A-10B illustrates one non-limiting embodiment of a de-focusing lens for varying a beam profile diameter of the laser system.

FIG. 11A-11B illustrates another non-limiting embodiment of a focusing lens for varying a beam profile diameter of the laser system.

### DETAILED DESCRIPTION

In the Brief Summary of the disclosure above and in the Detailed Description of the Disclosure described herein, and the claims below, and in the accompanying drawings, reference is made to particular features (including method steps) of the disclosure described herein. It is to be understood that the disclosure of the disclosure described herein in this specification includes all possible combinations of such particular features. For example, where a particular feature is disclosed in the context of a particular aspect or embodiment of the disclosure described herein, or a particular claim, that feature can also be used, to the extent possible, in combination with and/or in the context of other particular aspects and embodiments of the disclosure described herein, and in the disclosure described herein generally.

The embodiments set forth below re the necessary information to enable those skilled in the art to practice the disclosure described herein and illustrate the best mode of practicing the disclosure described herein. In addition, the disclosure described herein does not require that all the advantageous features and all the advantages need to be incorporated into every embodiment of the disclosure described herein.

In one embodiment, the laser therapy of the disclosure described herein is intended to non-invasively and non-ablatively treat soft tissue or hard tissue, such as cartilages, for acute or chronic inflammation or wounds. The method can include utilizing a laser or optical energy source and output device to irradiate inflamed or damaged cells in a wound. In one embodiment, the laser device can include a hand-held unit or hand piece that is made to be portable; however, it is contemplated within the scope of the disclosure described herein that the laser device may also be a fixed or mobile unit having a plurality of components. For example, a mounting structure can be used such that the hand piece may be used from a fixed position. In a further embodiment of the disclosure described herein, there may be multiple sequential surface area irradiations in a uniform, discrete, continuous, pulsed manner, or a combination thereof, to expose the maximum number of underlying inflamed or affected “reactive” cells. This sequential irradiation method can further include major blood cell concentrations in vascular structures as well as specific organ sites. Here, special attention can be directed to the irradiation of all or specific surrounding structures around acute or chronic inflammatory processes, including but not restricted to chronic ulcers, decubitus ulcers, diabetes related ulcers, acne vulgris certain types of psoriasis and acute or chronic abscesses. In addition, such treatment methods by the laser device of the disclosure described herein can be manual, automated, or pre-programmed. For example, a health practitioner may manually operate and direct the laser beam of the laser device on one or more areas to be treated. Alternatively, the laser device may automatically be pre-programmed to operate, or move about, to direct the laser beam on one or more areas of a patient for specific predefined time periods, continuous or pulsed operation, wavelengths, beam profiles, and power outputs, among others.

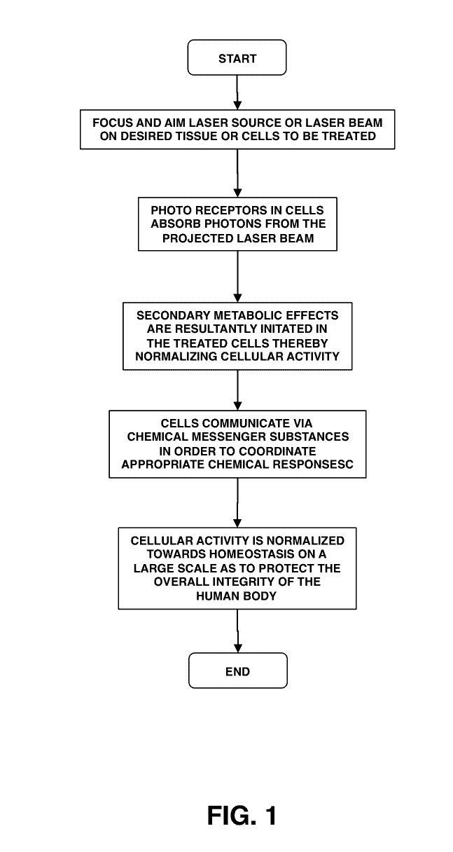

In reference to FIG. 1, in the disclosure described herein, laser irradiation is utilized to alleviate the physical symptoms associated with acute or chronic inflammatory conditions or wound healing. For example, symptoms such as pain and discomfort and specific organ involvement are the result of acute or chronic inflammation or infection of a wound. In one embodiment of the disclosure described herein, photo activation of intracellular photoreceptors initiates a cascade of secondary cellular metabolic effects, normalizing cellular activity towards homeostasis. This homeostasis is a fragile balance related to the reactive condition of adjacent cells. It is preferred to treat as many reactive or viable cells as possible at or near the same time interval in or near the wound area so as to generate a preponderance of neutral or homeostatic cell responses en masse. Further, the amount of time and intensity of treatment can be determined by the character of the cells to be treated, the depth of penetration desired, the chronicity of the condition, and the physical condition of the patient. Any number of factors in addition to those described above may be used to determine the operating levels of the hand piece such that it is operated below the photo ablation threshold of the tissue. The use of the laser therapy of the disclosure described herein acts to stimulate cellular regeneration, stabilize cell membranes, stabilize the indices of red blood cell deformation, increase lymphocyte counts, stimulate intracellular metabolism through mitochondrial photoreceptors, and stimulate the production of intercellular messenger proteins and enzymes, specifically superoxide dismutase and catalase enzymes. Additionally, there is immediate increase in membrane permeability of nerve cells and regeneration of Schwann cells lining the nerves. RNA and subsequently DNA production is enhanced. Singlet 02 is also produced which further contributes to cellular regeneration.

When these responses are exaggerated or erroneous as in the case of acute or chronic inflammation a violent cascade of cellular reactions contributes to biological changes which result in ongoing messenger signaling and elicits ongoing reactive cellular metabolic responses. Here, the rapid communication between immune and body cells brings about a proliferation of specific cell types within the inflamed area or its margins and orchestrate the various stages of anti-inflammatory processes through photo activation of cellular photoreceptors of large masses of cells a homeostatic intracellular metabolism and messaging. As intercellular messaging indicates homeostatic status, the reactive status of cells stabilizes through the various intracellular secondary metabolic effects and normalcy resumes.

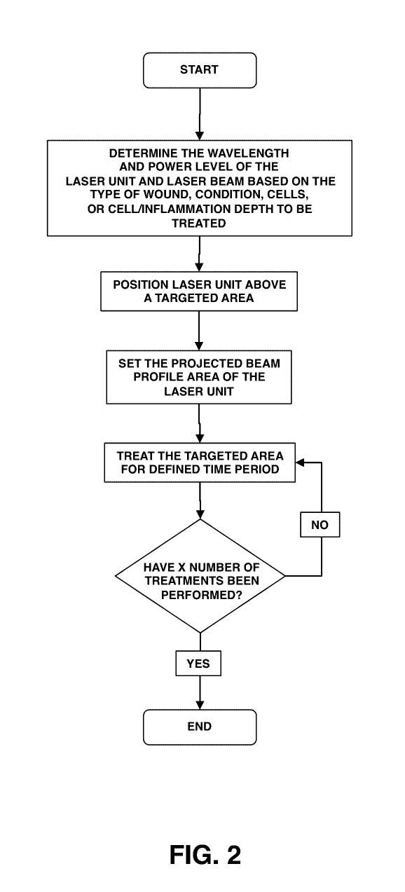

In reference to FIG. 2, in one embodiment, the hand piece or laser device is directed at damaged or inflamed cells in the wound and the laser device is configured to the appropriate wavelength and power level. Here, the wavelength, power level, beam profile, duration, and other operating parameters of the laser of the hand piece can depend on the depth and type of cells being treated, among other factors. Other factors can include but are not limited to the skin type, melanin levels, ethnical background/ancestry of the patient, past conditions, prior medical history, severity of the wound or inflammation condition, medications taken, among others. Here, laser or optical energy of the laser device and hand piece of the disclosure described here can be ideally operated in the infrared spectrum at wavelengths ranging from 1060 nm up to and including 1325 nm. However, it is contemplated within the scope of the disclosure described herein that the laser device or hand piece may be operated at any other wavelength. In addition, the laser or optical energy of the laser device or hand piece of the disclosure described herein can be operated at any level of power, with preference given to the 750 mW to 2.8 Watts/cm² range and/or 500 mW to 5 Watts/cm² range. However, it is contemplated within the scope of the disclosure described herein that the laser device or hand piece may also be operated at any power level. For example, in embodiment of the disclosure described herein, the hand piece and laser device can be operated at a wavelength of 1275 nm or 1064 nm and within the 750 mW to 2.8 Watts/cm² range.

In other embodiments, the laser device may be operated at or the laser therapy treatment method parameters include but not limited to any one or more wavelengths from 1060 nm to 1325 nm, at any one or more power levels from 500 mW/cm² to 5 Watts/cm², at any time duration from 5 seconds up to 3600 seconds, continuous or pulsed laser operation, at one or more beam profiles from an area of 0.1 cm² to 60 cm² or 0.1 cm to 60 cm in diameter, or any combination of the parameters thereof, to treat cells from any one or more depths of 1 cm to 30 cm. Here, the hand held laser can also be configured to operate in a continuous or pulsed mode at any of the aforementioned wavelengths, power levels, and beam profiles. In addition, the laser device or hand piece can have a homogenous beam profile between one square centimeter and sixty square centimeters of surface area irradiation. Here, the area of surface irradiation plays an important role to the efficient radiation of a high volume of cells concurrently. As previously noted it is felt necessary to overcome cell numbers in a “reactive” state to benefit from the secondary benefits of photo stimulation involving chemical messaging between cells. The projected beam may also be non-homogenous and may have a projected surface area less than or greater than the range described above. Further, in one embodiment of the disclosure described herein, the treatment duration range for a single treatment session can between 30 seconds to 3600 seconds, or from 3 hours up to 24 hours. However, it is also possible for the treatment duration to be shorter or longer.

For each treatment, the hand piece or laser device can be configured such that it is operated below the photo ablation threshold of the tissue being treated. Photo stimulation through the use of the laser device and method of the disclosure described herein at the disclosed parameters of wavelengths, power levels, beam profiles, and durations, among others, specifically activates the photoreceptors of cell membranes. This initiates Adenosine-tri-phosphate (ATP) production in the mitochondria of these reactive or viable cells. The increased cellular energy in the form of ATP is then used by the cell to finance cellular metabolic needs as well as other cellular functions such as angiogenesis, cellular regeneration, increase fibrosis and stimulation of the production of intercellular messenger proteins and enzymes as the cells moves metabolically towards homeostasis which is determined by genetic determination of cell type and function. In addition, the homeostatic cells can continuously communicate with adjacent and even distant cells by sending and receiving chemical messenger substances. These messenger substances relate cell status to adjacent and distant cells and coordinate appropriate chemical responses to protect the integrity of the body overall.

Further, platelets in a wound and surrounding area can produce platelet-derived growth factor (PDGF) that stimulates fibroblast proliferation during early as well as late phase of wound healing by promoting collagenase production from fibroblasts for wound remodeling that results in decrease or minimal scarring. This homeostasis is a fragile balance related to the reactive condition of adjacent cells. It is preferred to treat as many reactive or viable cells as possible at or near the same time interval in or near the wound so as to generate a preponderance of neutral or homeostatic cell responses en masse. In one embodiment, the amount of time and intensity of treatment is determined by the character of the cells to be treated, the depth of penetration desired, the chronicity of the wound, and the physical condition and age of the patient. Any number of factors in addition to those described above may be used to determine the operating levels of the hand piece such that it is operated below the photo ablation threshold of the tissue.

In one embodiment, the use of high powered non-invasive optical energy in the disclosure described herein acts to treat acute or chronic wounds through increase microcirculation, cellular regeneration, stabilize cell membranes, stabilize the indices of red blood cell deformation, increase fibrosis, stimulate intracellular metabolism through mitochondrial photoreceptors, and stimulate the production of intercellular messenger proteins and enzymes. Further, there can be an immediate increase in membrane permeability of nerve cells and regeneration of Schwann cells lining the nerves. RNA and subsequently DNA production is enhanced. All these enhanced physiological events lead to accelerated wound closure rates, increased tensile strength, and decrease or minimal scarring. Here, cellular responses in the case of an acute or chronic wound bring about a violent cascade of cellular reactions that contributes to biological changes which further result in ongoing messenger signaling and elicits ongoing reactive cellular metabolic responses. The rapid communication between immune and body cells brings about a proliferation of specific cell types within the wound or at the wound margins and orchestrate the various stages of wound healing through photoactivation of cellular photoreceptors of large masses of cells a homeostatic intracellular metabolism and messaging. As intercellular messaging indicates homeostatic status, the reactive status of cells stabilizes through the various intracellular secondary metabolic effects and normalcy resumes among cellular function.

Further, additional surface points of irradiation may also overlay cellular structures involved in cellular energy deficits secondary to or directly resulting from involvement in auto immune and immune mediated inflammatory reactions. These include but are not restricted to arterial endothelial cells, thyroid gland cells, pancreatic cells, liver cells, intestinal mucosal cells, brain cells and meninges, nerve cells, nerve ganglia cells, spinal cord cells, muscle cells, bone cells, cartilage cells, connective tissue cells, specialized respiratory cells, fat cells, and mucosal cells. These surface areas overlying the reactive cells may be irradiated concurrently or sequentially.

When immune system cells are actively engaged in creating antigen antibody reactions they release chemical messengers. These chemical messengers create the inflammatory cascade involving many other immune system cells that res the classic immune system reaction to foreign substances. This cascade of the immune system response also involves local cell types as the inflammatory response engulfs an area. This involvement in a classical immune system reaction is in the form of energy depleting to the cells involve, while intracellular metabolism is shifted away from homeostasis toward messenger instruction mediated reactivity. Long term resolution of cascaded immune inflammatory reactions requires stopping the cascade inflammatory stimulation while addressing the energy deficit of cells already impacted by the immune messenger chemicals. Photo stimulation of cells supplies energy for resumption of normal homeostatic cell metabolism, which in turn involves the release of chemical messengers directing adjacent and distant cells toward biological equilibrium or homeostasis.

Specific immune system cellular reactions can be treated in situ at the point of immune mediated inflammation with appropriate time and dose related treatments. In addition the preferred method specifies the in situ irradiation of areas of high concentration of vascular structures containing mobile immune cells whose metabolic status may be of a reactive nature. Irradiation of vascular structures in a time related and dose specific fashion with a wavelength that is capable of penetrating to the depth of a large volume of vascular structures is best to irradiate the largest number of cells within those structures.

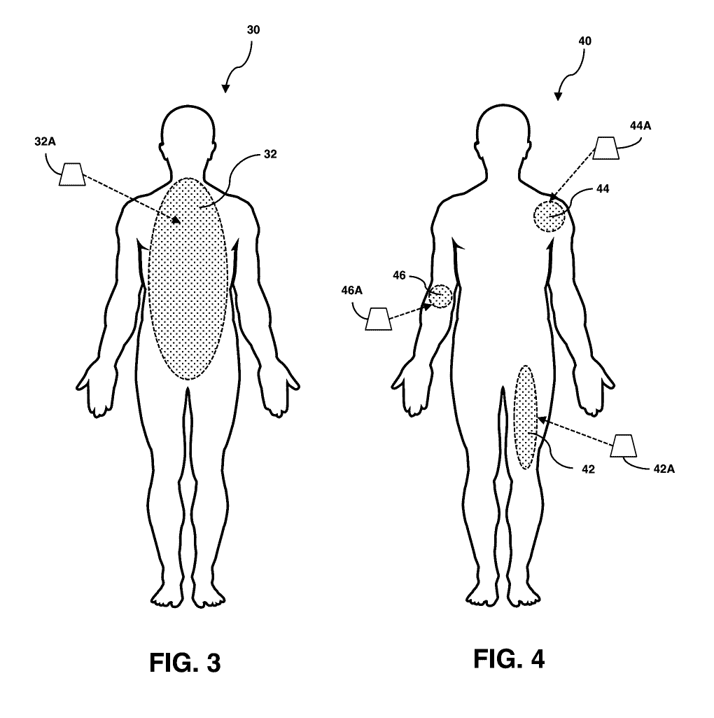

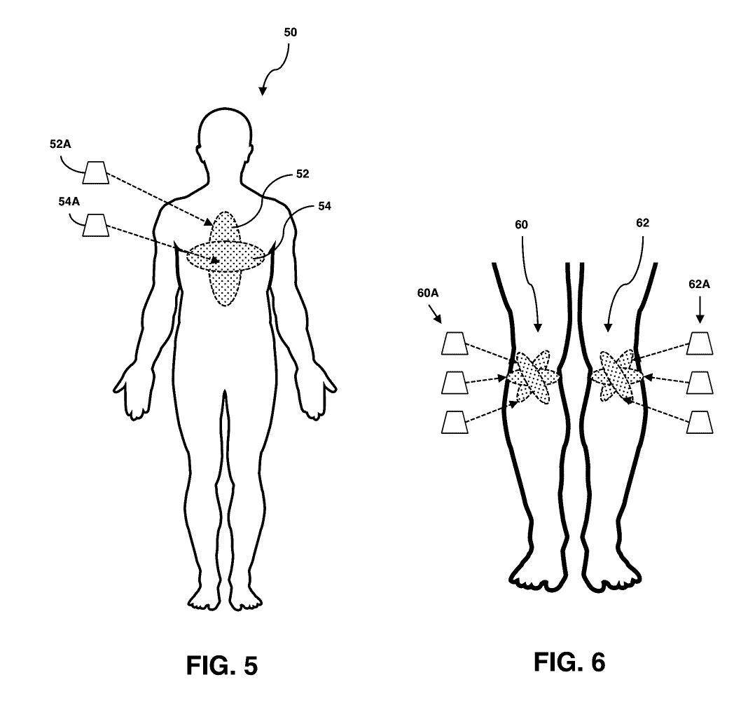

In reference to FIGS. 3-6, there may be one or plurality of hand pieces or laser devices used concurrently to irradiate acute or chronically inflamed cells in a wound as well as cells within the bloodstream moving through key vascular areas of high blood cell concentration. For example, referring to the embodiment of FIG. 3, there can be a single source or hand piece 32A directed a laser beam having a large surface beam profile 32 for treating one or more of wound, inflammation, or autoimmune deficiency conditions in patient 30. Referring to the embodiment of FIG. 4, there can be a plurality of laser sources such as 44A having beam profile 44, laser source 46A having beam profile 46, and laser source 42A having beam profile 42, wherein each laser source or hand piece can treat various areas, parts, and treatments sites of the patient's body 40. Referring to the embodiment of FIG. 5, there can at least two laser sources 52A and 54A with their beams 52 and 54 directed at the same treatment site area of patient 50. Here, by overlapping the projected conical distribution of laser light to coordinate with the depth of cells, the overlapping laser beams can utilize deep penetration of the 1060 nm to 1325 nm wavelengths, such as 1275 nm wavelength, and high power densities maintained below the level of cellular ablation, to increase the density of photon concentration to deep body cells. Similarly, FIG. 6 illustrates another embodiment wherein at least three laser sources 60A for one treatment site 60 and at least three laser sources 60B for another treatment site 62 can be utilized to further increase the photon concentrations at those sites for deeper penetration of the 1060 nm to 1325 nm wavelengths of the disclosure described herein. Further, the laser source, laser beam, or aimed optical energy can be either adjacent, in direct contact, indirect contact, near, in proximity, or at any distance with respect to the patient or treatment site.

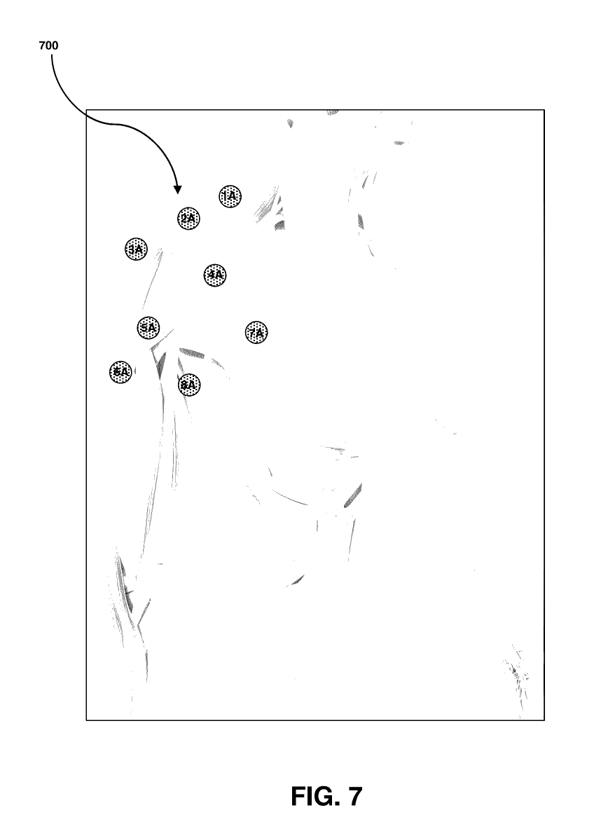

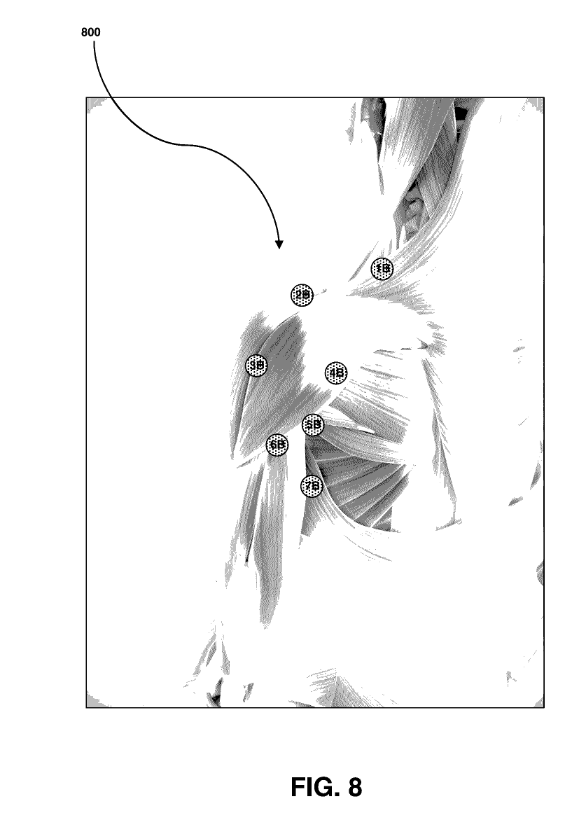

In reference to FIG. 7-8, an embodiment for a treatment protocol of the laser system, apparatus, and method of the disclosure described herein is provided. One embodiment for a treatment protocol of the laser unit of the disclosure described herein can be determining the site of an injury, wound, or inflammation, establish a grid at the injury site to determine the treatment points, set the laser to an appropriate power, and treat each point for approximately 60 seconds. Referring to FIG. 7, a perspective view of an anterior shoulder area 700 is illustrated. Here, the wavelength for the laser source or laser beam can be set to anywhere from 1064 to 1325 nm, and the power level set to 750 to 1200 mW/cm², wherein each treatment point 1A-8A is treated by the laser beam for approximately 60 seconds. Referring to FIG. 8, a perspective view of a posterior shoulder area 800 is illustrated. Similarly, the laser beam can be set to anywhere from 1064 to 1325 nm, and the power level set to 750 to 1200 mW/cm², wherein each treatment point 1B-7B is treated by the laser beam for approximately 60 seconds.

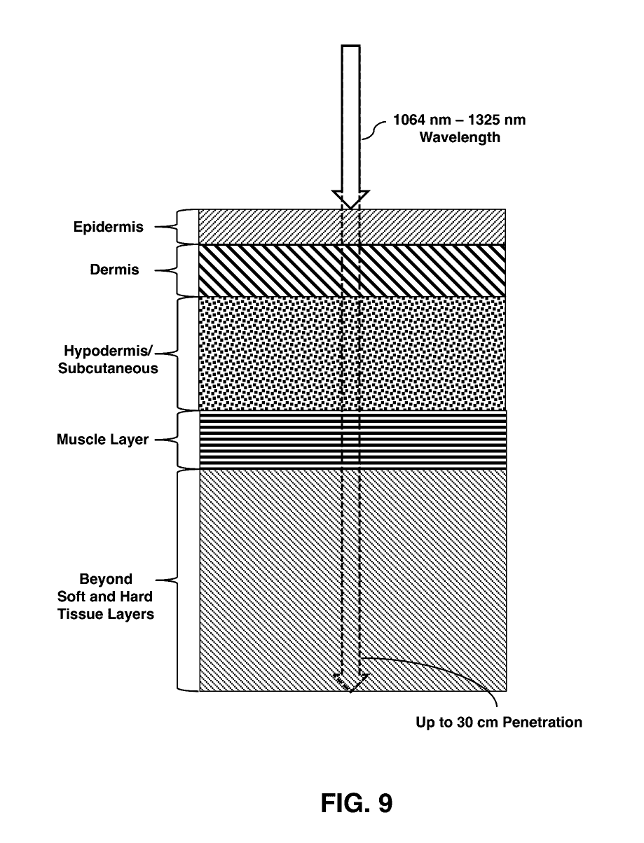

In reference to FIG. 9, a cross-sectional view of the various layers of tissue are illustrated, wherein the wavelengths of 1064 to 1325 nm of the disclosure described herein can penetrate deep within soft and hard tissue well beyond the muscle layer, such as up to 30 cm. In addition, it is contemplated within the scope of the disclosure described herein that the penetration depth can also be controlled via a combination of one or more of the range or specified wavelengths, power levels, and beam profiles disclosed herein, among other factors.

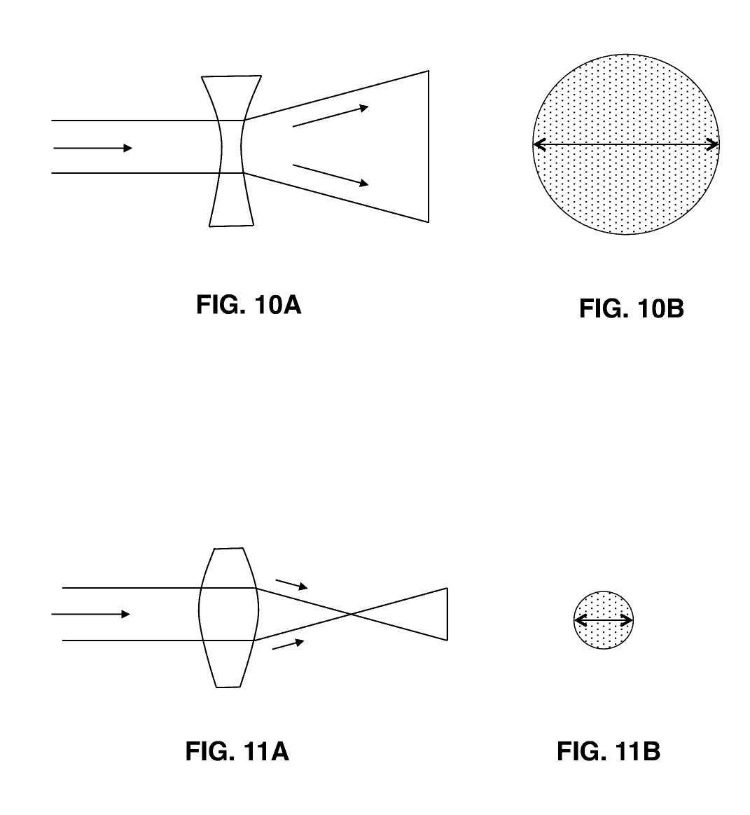

In reference to FIG. 10A-FIG. 11B, various beam profiles can be achieved can be achieved depending on the focusing and de-focusing of the laser source. For example, FIG. 10A and 10B illustrate one embodiment of de-focusing of the laser beam wherein a beam profile having a large can be achieved, such as up to 60 cm. FIG. 11A and 11B illustrate one embodiment of focusing the laser beam to achieve smaller diameters for the beam profile. However, it is contemplated within the scope of the disclosure described herein that any type of focused or de-focused laser beam or infrared light can be used to achieve a specified beam profile diameter or beam profile surface area. Further, the beam profile surface area of the laser source can also be altered or modified dynamically during a treatment session or previously defined or fixed. In addition, the laser source may use any type of lens to achieve a desired beam profile, including but not limited to concave, convex, biconvex, plano-convex, plano-concave, biconcave, meniscus, and doublet, or a combination thereof. In addition, the lenses may be cylindrical, astigmatic, aspheric, achromatic, and have any type of coating.

The laser system, method, and apparatus of the disclosure described herein advocates the use of a homogenous or non-homogenous beam of 1060 to 1325 nm coherent laser light or optical energy beam consistent with that of the laser. The disclosure described herein claims benefits from the use of between 1060 nm and 1325 nm wavelengths of infrared light. For example, one treatment method can specifically use a pre-defined 1275 nm, and another method can vary and set the wavelengths from approximately 1150 nm to 1200 nm, another method can have a fixed or dynamically varying wavelengths from 1100 nm to 1325 nm, wherein such wavelengths can be used with laser magnitude or power levels of from 750 nm to 2.8 Watts/cm² range. The disclosure described herein claims deep tissue penetration from 1060 to 1325 nm wavelength laser light in accordance with established models which illustrate preferable low absorption rates in melena, hemoglobin, and water at 1060 to 1325 nm wavelengths and more specifically at 1100-1150 nm or up to 1325 nm with power levels from 750 mW/cm² to 2.8 Watts/cm² range and beam profile diameter profiles that can range from 1 inch up to 20 inches in a continuous wave. Here, the continuous wave or continuous waveform (CW) laser operation of the present disclosure described herein produces a continuous output beam, as opposed to pulsed operation. Here, the continuous wave can allow for far greater energy delivered in a shorter period of time and greater wattage and area covered by the laser allows for more coverage of surface tissue in a much shorter period of time than pulsed mode aser therapies. Further, it is contemplated within the scope of the disclosure described herein that the laser may include one or more diodes and in addition to or in lieu of continuous wave (CW) operation, it may also operate in a quasi-continuous wave operation model, wherein pump source is switched on only for certain time intervals, which are short enough to reduce thermal effects significantly, but still long enough that the laser process is close to its steady state.

The disclosure described herein claims that larger homogenous beam profile accompanied by higher power source results in larger total three-dimensional areas of cells in a wound to be irradiated. Cells are irradiated at powers less than that of cellular ablation. The disclosure described herein claims that only through high powers, wide beam profile and specific wavelengths can the largest number of inflamed cells can be irradiated concurrently. The disclosure described herein claims that concurrent irradiation of large numbers of cells initiates' photo activation of preceptors in all cells irradiated in or around the inflamed wound margins. The disclosure described herein claims that concurrent activation of photoreceptors in cells, cell metabolism moves toward physiologic equilibrium concurrently. The disclosure described herein claims that secondary cell functions occur as a result of stimulation of cellular photoreceptors and the concurrent enhancement of cellular energy status as well as the resumption of more normal cellular metabolic activity is the basis for anti-inflammatory processes initiated by specifically using either the 1275 nm or 1064 nm with power levels from 500 mW to 5 Watts/cm² range.

The following conditions may also be treated with the optical irradiation therapy of the disclosure described herein, including but not limited to: inflammatory arthritis Rheumatoid arthritis; Ankylosing spondylitis, Sjogren syndrome, Osteoarthritis (degenerative joint disease), Knees, Thumb, Cervical spine, Lumbar spine, Perianritis (“frozen shoulder”), Tendonitis, Lateral epicondylitis (tennis elbow), Medial epicondylitis, Supraspinatus tendonitis, Bicipital tendinitis, Achilles tendinitis, Neuropathic pain, Carpal tunnel syndrome, Diabetic neuropathy, Radiculopathy, Radiation dermatitis, Stomatitis, Keioids, Sports injuries, Ankle sprain, Muscle pulls, Buerger's disease (Thromboangiitis Obliterans), Headaches (vascular and muscular), Pruritus, Peripheral nerve repair, Post therapeutic, Neuralgia, Oro-facial pain, Dental surgery, Oral dysesthesia, Trigeminal neuralgia, Temporomandibular pain, Dental hypersensitivity, Acute and chronic musculoskeletal pain, Low back pain, Tension myalgia, Myofascial pain, Patellofemoral pain, Soft tissue wounds, Diabetic ulcers, Pressure sores, Venous stasis ulcers, Surgical Wounds, Following neurosurgery, Cranial nerve VII (facial nerve) repair, Trigger point (elevation of pain thresholds), Sympathetic nervous system dysfunction, Hemangioma, Tinnitusimmune modulation, Allergetic rhinitis, Leukemia, Bactericidal effects, and Pyronie's disease Nerve repair, among others.

Further, the types of lasers that may be used for method, system and apparatus of the disclosure herein may include, but is not limited to: Helium Neon (NeBe), Gallium aluminum arsenide (GaAlAs), Gailium arsenide (GaAs), Neodumium-yttrim-alumimum gamet (Nd:YAG), Carbon dioxide (CO₂), Argon (Ar), Krypton (Kr), Ruby, and Diode, among others.

Further, in one embodiment for a treatment action of the laser therapy system disclosed herein can include acute inflammation reduction. More specifically, immediately after an acute injury event, the body, in response to the disruption of the integrity of vascular, soft tissue, connective tissue and neurological processes, initiates a series of biological responses. The inflammatory reaction can consist of both vascular and cellular events. Here, injury responsive components such as Mast cells, Bradykinins and Prostaglandins are activated along with the vascular responses and cellular membrane reactions. All of these combined processes and events are represented by the symptoms of edema, inflammation, pain and functional debility. Laser light therapy of the present disclosure described herein can be effective in mediating both the aforementioned symptoms and the underlying inflammatory process. Here, the laser light energy pulses of the disclosure described herein can be adjusted to penetrate more deeply and more aggressively into the skin tissue, depending on the condition and goals of treatment. The light energy, which can be delivered by either a large device that emits multiple laser panels at once, or a hand-held device for smaller targeted areas, which will pass through the skin layers to reach the cells and tissue causing the pain and inflammation. Here, the laser device can also be held against the skin over the area being treated, and the light energy is absorbed and converted to biochemical energy which stimulates the cells. The activity activates the natural healing process of the cells, which reduces pain, increases blood flow, and stimulates repair of the tissue.

In another embodiment for a treatment action of the laser therapy system disclosed herein can include targeting inflammation. More specifically, for inflammation, the laser therapy of the disclosure described herein can cause the smaller arteries and lymph vessels of the body to increase in size, which is called vasodilatation. Vasodilatation allows inflammation, swelling, and edema to be cleared away from injury sites more effectively. Vasodilatation in lymph nodes promotes lymphatic drainage which also aids in the healing process; bruises are also often resolved faster due to this effect.

In another embodiment for a treatment action of the laser therapy system disclosed herein can include management of fibromyalgia. More specifically, the therapeutic laser light energy of the disclosure described herein has good pain relieving and anti-inflammatory effects that provide considerable pain relief for patients with fibromyalgia (FM), and can significantly increase the quality of life for such patients. The laser light therapy of the disclosure described herein combination with other treatment modalities, such as medications, can offer another positive multidisciplinary approach to FM treatment.

In another embodiment for a treatment action of the laser therapy system disclosed herein can include back, neck, and joint pain management. In particular, there are a number of biochemical effects that have been observed with laser therapy, several of these effects relate directly to the management of the patient with chronic back pain. Three of the most prevalent features of patients suffering from chronic back pain are inflammation, pain, and edema. Further, injured cells and tissues generate enzymes that encourage the receipt of photons more readily than healthy cells and tissues do. Primary photo acceptors which are located in the mitochondria are activated by the laser light of the disclosure described herein and can convert the light energy into electrochemical energy. These are thought to be avins, cytochromes, and chromophores in the form of porphorins. Porphyrins have been shown to play an important role in the relief of low back pain. Small amounts of singlet oxygen have been shown to accumulate in tissues irradiated with laser light. Further, singlet oxygen affects the formation of adenosine-5′-triphosphate (ATP) in the mitochondria, and the red and infrared light therapy of the disclosure described herein can reduce pain by a combination of these responses. In particular, biochemical responses to the laser therapy of the disclosure described herein can include, but is not limited to: stabilization of the cell membrane, enhancement of ATP synthesis, stimulated vasodilatation along with increased histamine, nitrous oxide, and serotonin, acceleration of leukocyte activity, increased prostaglandin synthesis, reduction in interleukin-1 levels, Increased angiogenesis, enhanced superoxide dismutase, and decreased C-reactive protein and neopterin levels, among others.

In another embodiment for a treatment action of the laser therapy system disclosed herein can include neurologic response management. In particular, There are several neurologic responses to laser therapy that may influence brain recovery or prevent brain atrophy as well as several of the physiologic effects listed above. In particular, the application of laser to normal human neural progenitor (NHNP) cells can significantly increase ATP production. In addition, the laser therapy of the disclosure described herein has the potential to improve neuronal function in many patients with Parkinson's disease and other neurodegenerative diseases.

In another embodiment for a treatment action of the laser system disclosed herein can provide deep tissue penetration and saturation. In particular, chronic low back pain is a complex clinical condition that involves many different tissue levels from subcutaneous and muscle tissues to the deeper tendons and ligaments, including the intervertebral disc. The laser therapy of the disclosure described herein is effective in treating such pain in that it will produce significant biochemical changes in the superficial, medium, and deep tissues of the treatment site. In particular, the laser light energy of the disclosure described herein can affect deep tissue structures from approximately 1 cm to 30 cm in depth.

In another embodiment for a treatment action of the laser system disclosed herein can include wound care management. In general, laser light therapy is a form of phototherapy that involves the application of high power monochromatic and coherent light to injuries and lesions in order to stimulate wound healing. The laser light therapy of the disclosure described herein has been shown to increase the speed, quality and tensile strength of tissue repair, resolve inflammation and provide pain relief. In addition, during a laser irradiation session, cells absorb photonic energy of the laser system disclosed herein that is incorporated into chromophores, which, in turn, stimulates cellular metabolism. Hence, the effects are photochemical, not thermal, and the responses of cells occur due to changes in photo acceptor molecules (also known as chromophores, which are molecules that are able to absorb photonic energy such as porphyrin. Here, the chromophore is able to transfer the absorbed energy to other molecules and thus cause chemical reactions in surrounding tissue. Further, the acceptor molecules' kinetic energy is increased, thereby activating or deactivating enzymes, which, in turn, are able to alter the physical and/or chemical properties of other macromolecules, such as DNA and RNA in order to facilitate wound healing. Here, the light energy which is delivered to the cells produces insignificant and minimal temperature changes, such as in the range of 0.1 to 0.5 Celsius so that the treatment is essentially painless and non-ablative of the target tissue and surrounding tissue.

Although the disclosure described herein has been explained in relation to various embodiments, it is to be understood that many other possible modifications and variations can be made without departing from the spirit and scope of the disclosure described herein as here in after claimed.

1. A method of laser irradiation for alleviating the physical symptoms associated with acute or chronic inflammatory conditions comprised of:
focusing a light beam having a configured wavelength and power level on an inflamed area that is to be treated;
wherein the light beam photo activates intracellular photoreceptors, thereby initiating a cascade of secondary cellular metabolic effects and normalizing cellular activity towards homeostasis.

2. The method of claim 1, wherein the focusing further comprises treating surrounding reactive cells or viable cells near the inflamed area at identical time intervals so as to generate a preponderance of neutral or homeostatic cell responses en masse.

3. The method of claim 1, wherein the light beam generate an increase in oxygenation in or around the inflamed area margins through angionesis or revascularization leading to wound healing or homeostatic cell response en masse.

4. The method of claim 1, wherein the focusing further comprises stimulating the production of intercellular messenger proteins and enzymes including superoxide dismutase and catalase enzymes.

5. The method of claim 1, wherein the wavelength is further comprised of one or more wavelengths ranging from 1064 nm up to and including 1325 nm.

6. The method of claim 1, wherein the power level is further comprised of one or more power levels ranging from 500 mW/cm² up to and including 5 W/cm².

7. The method of claim 1, wherein the light beam further comprises a beam profile covering a surface area ranging from 0.1 cm² up to and including 60 cm².

8. The method of claim 1, wherein the light beam further comprises a duration period from 30 seconds up to and including 3600 seconds.

9. The method of claim 1, wherein the light beam is further comprised of continuous wave operation.

10. The method of claim 1, wherein the light beam does not ablate cells within the inflamed treatment area or surrounding tissue area.

11. A method of laser irradiation for alleviating the physical symptoms associated with acute or chronic inflammatory conditions, the method comprising:
positioning an optical source having infrared light adjacent to a treatment site;
directing the infrared light at the treatment site;
configuring a wavelength for the optical source, depending on the depth and type of inflammation at the treatment site; and
determining a power level for the optical source, wherein the infrared light photo activates intracellular photoreceptors at the treatment site.

12. The method of claim 10, wherein configuring wavelength further comprises configuring a wavelength in the infrared spectrum between from 1060 nm to 1325 nm.

13. The method of claim 10, further comprises configuring the optical source in power ranges from 750 mW/cm² to 1200 mW/cm².

14. The method of claim 10, further comprising configuring the optical source in power ranges from 350 mW/cm² to 1200 mW/cm².

15. The method of claim 10, further comprising configuring the optical source in power ranges from 500 mW/cm² to 5 Watts/cm².

16. The method of claim 10, wherein the optical source is further comprised of a hand-held unit.

17. A method of laser irradiation for alleviating the physical symptoms associated with acute or chronic inflammatory conditions, the method comprising:
directing a laser unit having infrared light at damaged or inflamed cells;
configuring the laser unit to a wavelength of 1100 nm to 1275 nm and a time duration, depending on the depth and type of cells to be treated, wherein the depth can range from 0.1 cm to 15 cm;
configuring the laser unit to a power level of 750 mW/cm² to 1200 mW mW/cm²; and
configuring the infrared light to a beam profile surface area of 1 cm² to 60 cm².

18. The method of claim 16, further comprising operating the laser unit in a continuous wave mode and having a homogenous beam profile.

19. The method of claim 17, further comprising treating the damaged or inflamed cells in a single treatment session for a duration of time ranging from 30 seconds to about 3600 seconds, and wherein the laser unit operates below the photo ablation threshold of the cells being treated.
